(19) United States
(12) Patent Application Publication
(10) Pub. No.: US 2009/0137996 A1
(43) Pub. Date: May 28, 2009

(54) NONABLATIVE AND ABLATIVE TISSUE TREATMENT METHOD AND DEVICE

(76) Inventor: Leonard C. DeBenedictis, Palo Alto, CA (US)

Correspondence Address: RELIANT / FENWICK;c/o FENWICK & WEST, LLP, 801 CALIFORNIA STREET, MOUNTAIN VIEW, CA, 94041, US

(21) Appl. No.: 12/324,359

(22) Filed: Nov. 26, 2008

Related U.S. Application Data
(60) Provisional application No. 60/990,881, filed on Nov. 28, 2007.

(51) Int. Cl. A61B 18/20; A61B 18/18

(52) U.S. Cl. 606/9; 606/33; 606/10

(57) ABSTRACT

Methods and devices for treatment of tissue which first apply a nonablative form of electromagnetic energy to a region of tissue to create a plurality of treatment zones containing coagulated tissue and subsequently apply an ablative form of electromagnetic energy to the coagulated tissue in the treatment zones in order to ablate the coagulated tissue are disclosed. These methods and devices can be used to shrink and/or tighten tissue for medical and cosmetic purposes.

### CROSS-REFERENCE TO RELATED APPLICATION(S)

This application claims priority under 35 U.S.C. §119(e) to U.S. Provisional Patent Application Ser. No. 60/990,881, “Nonablative and Ablative Tissue Treatment Method and Device,” filed Nov. 28, 2007 by Leonard C. DeBenedictis. The subject matter of the foregoing is incorporated herein by reference in its entirety.

### FIELD OF THE INVENTION

This invention relates generally to methods and devices for treatment of tissue which employ a combination of nonablative and ablative forms of electromagnetic energy. More particularly, it relates to methods and devices for treatment of tissue which first apply a nonablative form of electromagnetic energy to a treatment site to coagulate tissue and subsequently apply an ablative form of electromagnetic energy to the same treatment site to ablate the coagulated tissue.

### BACKGROUND

Tissues such as, for example, human skin, often lose their elasticity due to chronological and/or photoaging, making it desirable to shrink the tissues to restore a more youthful and/or aesthetically pleasing appearance. Various forms of electromagnetic energy have been used to treat tissue in order to resurface, rejuvenate, tighten and/or shrink the tissue. These electromagnetic energy-based treatments can be broadly classified into two types of treatments: ablative and nonablative treatments. Ablative electromagnetic energy-based treatments result in the bulk removal of tissue from the treatment site. Nonablative treatments produce thermal effects in the tissue at the treatment site, such as, for example, necrosis and/or coagulation of the tissue, but do not result in the bulk removal of tissue from the treatment site. Traditionally, methods and devices for treating tissue have used only one form or wavelength of electromagnetic energy to treat a region of tissue. When the correct treatment parameters are used, these treatments can produce good results, such as reducing the appearance of wrinkles and providing a moderate level of tissue shrinkage.

Methods and devices which employ two or more forms of or wavelengths of electromagnetic radiation have previously been used for purposes such as, for example, to provide illumination or a visual aiming beam for a treatment beam that is not in the visible spectrum, to simultaneously both ablate and coagulate a treatment area, to simultaneously heat and treat a treatment area, and the like. An example of these types of devices includes the device described in U.S. Pat. No. 4,638,800; a laser beam surgical system comprising a white light source to illuminate the surgical site coupled to a carbon dioxide laser to treat the surgical site. Another example of these types of devices includes the device described in U.S. Pat. No. 6,702,808; a system for applying, essentially simultaneously, radiofrequency (RF) energy and optical energy to skin. Yet another example of this type of device includes the device described in U.S. Pat. No. 4,791,927; a dual-wavelength laser system with both cutting and coagulating capabilities. An example of these types of methods includes the method described in U.S. Pat. No. 5,304,167; a method for simultaneously transmitting and delivering to a tissue site at least two wavelengths of therapeutic radiant energy along a common optical pathway, which allows a physician to simultaneously ablate and coagulate tissue using two wavelengths of radiant energy.

The methods and devices described above which both ablate and coagulate using two or more forms or wavelengths of electromagnetic radiation have been focused either on first ablating and then coagulating tissue to stop bleeding, or on simultaneously ablating and cutting in order to minimize or prevent bleeding. The processes of first ablating and then coagulating tissue, or simultaneously ablating and coagulating tissue, are ideal for surgical applications but do not produce high levels of tissue shrinkage, as are desired for the purposes of tightening skin, reducing the appearance of wrinkles, or rejuvenating skin.

### SUMMARY OF THE INVENTION

The present invention is directed to method and devices for shrinking and/or tightening tissue such as, for example, skin. The method comprises the steps of selecting a region of tissue in need of tightening, first treating the region of tissue using a nonablative form of electromagnetic energy in a manner so as to coagulate tissue within a plurality of treatment zones in the region of tissue; subsequently treating the region of tissue using an ablative form of electromagnetic energy in a manner so as to ablate at least a portion of the coagulated tissue within at least a portion of the plurality of treatment zones in the region of tissue while substantially avoiding ablating uncoagulated tissue in the region of tissue, whereby the first and subsequent treatings shrink and tighten the region of tissue. In one example, the subsequent ablative treatment or treatments can be conducted immediately following the first coagulative treatment. In another example, the first and subsequent treatments can be provided in the same treatment session. In yet another example, the first coagulative treatment can be conducted in a first treatment session, and the subsequent ablative treatment or treatments can be conducted in a later treatment session. In another example, there can be a delay between the first and subsequent treatment or treatments, wherein the delay does not exceed the duration of time required for the tissue to heal and substantially replace the tissue coagulated in the first treatment.

The present invention includes a device for shrinking and/or tightening tissue. One embodiment of the device comprises at least one source of nonablative electromagnetic energy configured to apply the energy in a manner so as to coagulate tissue in a plurality of treatment zones in a region of tissue, at least one source of ablative electromagnetic energy configured to apply the energy in a manner so as to ablate at least a portion of the coagulated tissue from at least a portion of the plurality of treatment zones in a region of tissue, and a controller configured to control the sources of electromagnetic energy. Another embodiment of the device further comprises a detector configured to detect the location and/or presence of coagulated tissue in the region of tissue and provide feedback to the controller, wherein the controller uses the feedback from the detector to control the ablative electromagnetic energy source in order to apply the ablative form of electromagnetic energy to the treatment zones in order to ablate at least a portion of coagulated tissue from at least a portion of the treatment zones without ablating substantial portions of uncoagulated tissue in order to shrink and/or tighten the region of tissue.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a series of four drawings (1A, 1B, 1C and 1D) illustrating the effects of an electromagnetic energy-based treatments which produce a plurality of treatment zones in a region of tissue.

FIG. 2 is a series of four drawings (2A, 2B, 2C and 2D) illustrating the effects of various examples of treatments where nonablative and ablative treatment zones of various sizes and patterns have been produced in a region of tissue.

FIG. 3 is a series of two drawings (3A and 3B) illustrating an example of a treatment device configured to deliver a first nonablative electromagnetic energy-based treatment followed by a second ablative electromagnetic energy-based treatment.

### DETAILED DESCRIPTION OF THE PREFERRED EMBODIMENTS

Fractional treatment methods involve the generation of a large number of treatment zones within a region of tissue. In fractional electromagnetic energy-based treatments, the energy impacts directly on only a number of relatively small zones of tissue within a larger region of tissue, instead of impacting directly on all of the larger region of tissue undergoing treatment, as it does in conventional bulk treatment methods. Thus, a region of skin treated using electromagnetic energy delivered in a fractional manner contains a plurality of treatment zones where the tissue has been exposed to the energy within a larger volume of tissue that has not been exposed to the energy. Fractional treatment methods make it possible to leave substantial volumes of tissue unaltered and viable within a region of tissue undergoing treatment.

Fractional treatment methods have been used to provide effective treatments for both treatment of existing medical (e.g., dermatological) disease conditions and for treatment aimed at improving the appearance of tissue (e.g., skin) by intentionally generating zones of thermally altered tissue surrounded by untreated tissue. Fractional treatment methods offer numerous advantages over existing approaches in terms of safety and efficacy, as they minimize the undesirable side effects of pain, erythema, swelling, fluid loss, prolonged reepithelialization, infection, and blistering generally associated with bulk optical energy based treatments of tissue. By sparing healthy tissue around the treatment zones, fractional treatment methods increase the rate of recovery of the treatment zones by stimulating remodeling and wound repair mechanisms. Fractional treatment methods also reduce or eliminate the side effects of repeated electromagnetic energy treatments to tissue by controlling the extent of tissue necrosis due to exposure to the energy.

Treating tissue with electromagnetic energy can produce many different types of effects in the tissue, including denaturation, coagulation, cell necrosis, melting, welding, retraction, alteration of the extra-cellular matrix, charring, and ablation. The type of effect or effects produced in the tissue, the depth to which the effect or effects extend into in the tissue, as well as the diameter of the zone of tissue affected by the energy, are dependent upon the treatment parameters used. These treatment parameters include the wavelength, the total irradiance, the local irradiance, the total fluence, the local fluence, the pulse energy, the pulse duration, the pulse repetition rate, the size of the treatment beam or electrode, the density of zones treated per square centimeter of tissue surface for fractional treatments, etc. The condition of the tissue (e.g., the hydration level of the tissue, the level of chromophores present in the tissue, etc.) can also affect the type of effect or effects produced in the tissue, the depth to which the effect or effects extend into the tissue, and the diameter of the zone of tissue affected by the energy.

Treatment of tissue with electromagnetic energy in a manner so as to cause thermal coagulation of the tissue, while causing necrosis of the coagulated zone, produces a thermal wound that can be rapidly repaired by the surrounding living tissue and, under many conditions, does not result in adverse effects, such as, for example, scarring or pigmentation changes in skin. Producing coagulated zones of tissue using fractional treatment methods can further reduce the incidence of adverse effects. Methods of using fractional photothermolysis to create microscopic lesions that allow for dermal content to be exfoliated through the stratum corneum are described, for example, in U.S. patent application Ser. No. 11/548,248, which is herein incorporated by reference.

The methods of the present invention include treating a region of tissue first with a form of nonablative electromagnetic energy to thermally coagulate the tissue within a plurality of treatment zones in the region of tissue, and subsequently treating the region with a form of ablative electromagnetic energy to ablate at least a portion of the thermally coagulated tissue from within the treatment zones, while leaving a portion of the tissue within the region untreated by either the first or the subsequent treatment, in order to tighten the entire region of tissue. By treating the tissue in this manner, the nonablative first treatment, by thermally coagulating treatment zones, thermally denatures collagen within the tissue of the treatment zones, altering the tertiary structure of the collagen and producing shrinkage of the collagen fibrils. The ablative second treatment is administered in a manner whereby the treatment ablates all or a portion of the tissue that was thermally coagulated by the first treatment, thus removing tissue that had previously been shrunken and tightened. By ablating coagulated tissue in the treatment zones, it is shrunken tissue that is removed, and the removal of the shrunken tissue allows the surrounding tissue to shrink even more, further tightening the tissue. Using an ablative wavelength that also provides some coagulative effect can yet further tighten the tissue. Such effects can be achieved using a CO₂ laser, as described in more detail, for example, in co-pending U.S. patent application Ser. No. 11/674,031, entitled Laser System for Treatment of Skin Laxity, which is herein incorporated by reference.

According to the present invention, the timing of the first and second treatment is such that the first treatment preferably has coagulated tissue in the treatment zones, and the coagulated tissue remains present in the region of tissue until the application of the second treatment. Therefore, the first and second treatments can be applied immediately following each other, or can be applied sequentially with a gap in time between them. Alternatively, the ablative treatment can begin after the nonablative treatment has begun to coagulate the tissue in the region undergoing treatment. The first nonablative treatment and the second ablative treatment can be each applied in one pass of the device or in one treatment session, or can each be applied in more than one pass of the device or in more than one treatment session.

In addition to producing higher levels of tissue tightening, the treatments of the present invention also produce less bleeding and oozing of fluid as compared to other fractional ablative treatments, as the portion of skin that is ablated has already been coagulated and thus is less prone to bleeding and oozing fluid. These treatments, while providing the principal benefits associated with fractional treatments, shrink and tighten the skin more than if either an ablative or a nonablative treatment were given alone. Further, as the ablated tissue is coagulated prior to ablation, it reduces the level of side-effects as compared to an ablative treatment when given alone. The difference in side effects between the treatment of the present invention and a solely ablative treatment is more significant when treatments providing equivalent levels of tissue shrinkage are compared. Yet further, when the first and subsequent treatments are provided in a time setting where the ablative treatment is provided immediately after the nonablative treatment, or where the ablative treatment is provided during the start of the coagulation of the tissue with the nonablative treatment, it is possible to provide such treatments using a device that can provide both treatments in one pass. The ability to provide both the nonablative and ablative treatments in one pass allows a significant level of shrinkage to occur in the region of tissue being treated in one pass, so that the overall nonablative and ablative treatment can be given rapidly and efficiently.

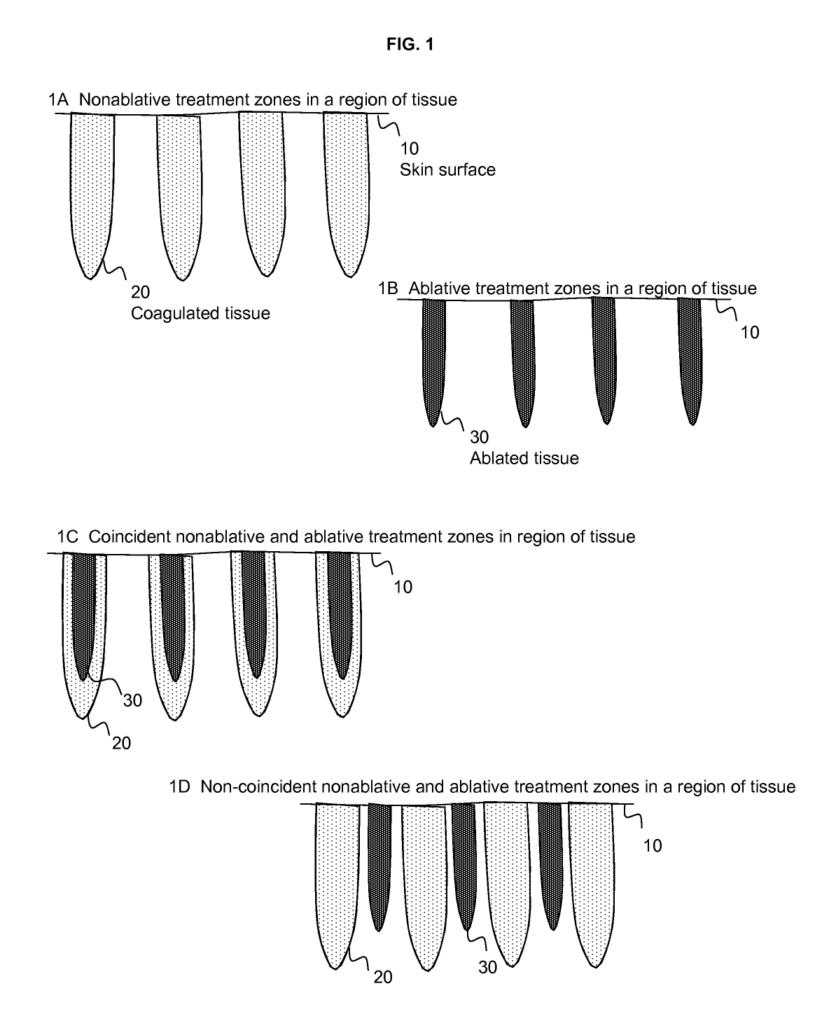

FIG. 1 is a series of four drawings illustrating the effects of electromagnetic energy-based treatments which produce a plurality of treatment zones in a region of tissue such as, for example, a skin surface 10. FIG. 1A illustrates treatment zones containing coagulated tissue 20 created by a nonablative treatment given alone. FIG. 1B illustrates treatment zones of ablated tissue 30 created by an ablative treatment given alone. FIG. 1C illustrates treatment zones created by a first nonablative treatment which coagulates tissue in the treatment zones 20, which is followed by a second ablative treatment which ablates at least a portion of the coagulated tissue from the treatment zones 10. This successive, coincident treatment first produces treatment zones containing coagulated tissue 20 and then ablates coagulated tissue 30, and thus minimizes the total surface area or volume of tissue exposed to treatment while producing high levels of tissue shrinkage and tightening in the treated region of tissue. FIG. 1D illustrates treatment zones created by a first nonablative treatment, which creates coagulation zones 20, followed by a second ablative treatment, which creates ablated zones 30. In this example, the two treatments are successive but are not coincident, and thus each treatment produces separate treatment zones (20 and 30), which increases the surface area and volume of tissue exposed to a treatment when compared to the treatment described in FIG. 1C because this treatment (FIG. 1D) ablates tissue 30 that had not been previously coagulated.

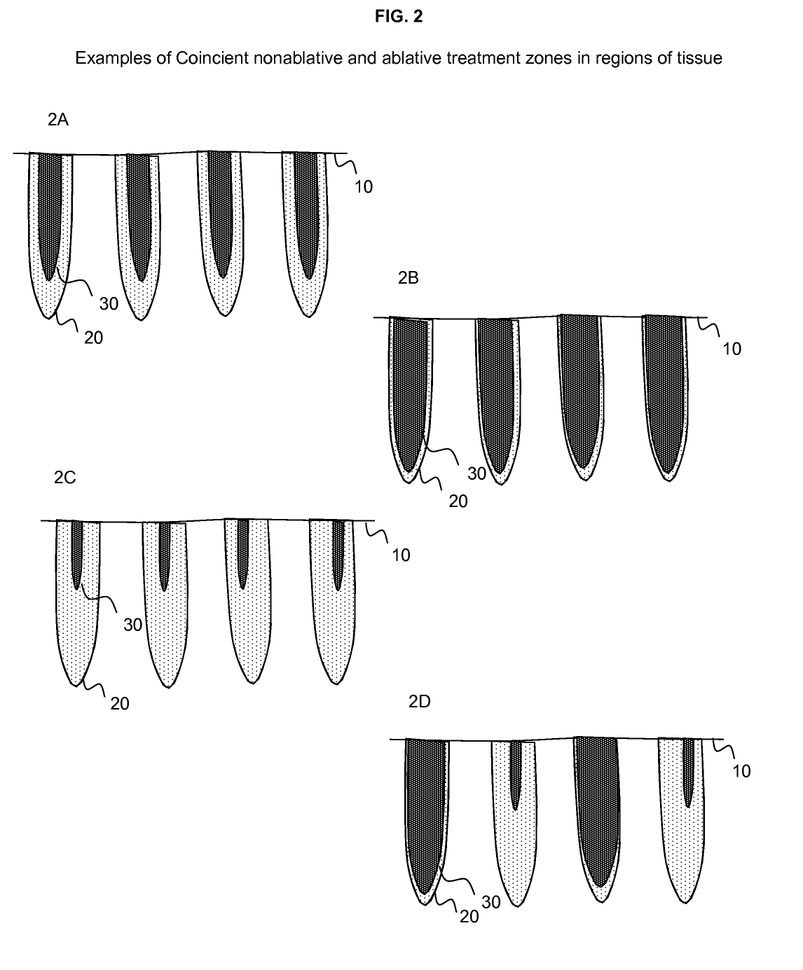

FIG. 2 is a series of four drawings illustrating various examples of treatment zones produced by successive coincident nonablative and ablative treatments on a skin surface 10. In FIG. 2A, the ablated treatment zones 30 are somewhat smaller than the coagulated treatment zones 20. In FIG. 2B, the ablated treatment zones 30 are approximately the same size as the coagulated treatment zones 20. In FIG. 2C, the ablated treatment zones 30 are significantly smaller than the coagulated treatment zones 20. In FIG. 2D, the coagulated treatment zones 20 are essentially uniform in size, but the ablated treatment zones 30 vary in size. The size of the ablated region of tissue can be controlled in order to provide more or less shrinkage or tightening in a region of tissue undergoing the combined nonablative and ablative treatment. Ablated zones 30 can also be larger than the coagulated treatment zones 20 and/or can partially overlap the coagulated treatment zones 20. By partially overlapping the treatment zones, the side effects associated with coverage area of the dermal-epidermal junction can be reduced in comparison to non-overlapping treatment zones. Thus, the first treatment can coagulate at least a portion of the epidermis/dermis and the second treatment can coagulate at least a portion of the coagulated epidermis/dermis within at least a portion of the treatment zones.

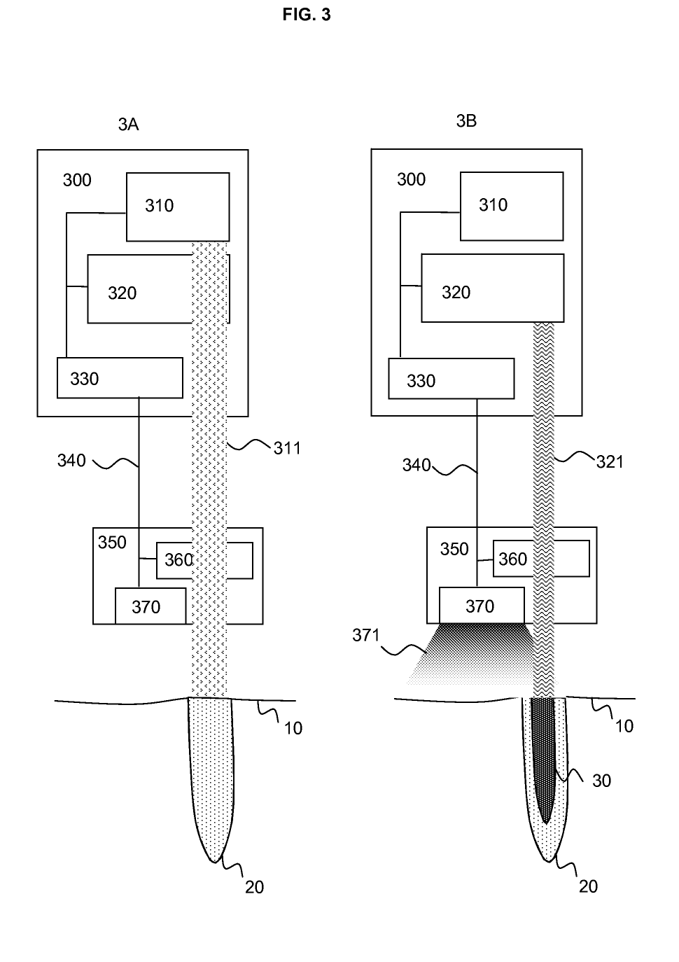

FIG. 3 is a series of two drawings (3A and 3B) illustrating an example of a treatment device in accordance with the present invention. The treatment device 300 comprises a first optical energy source 310, a second optical energy source 320, a controller 330, a handpiece 350, an optical energy delivery system 360 located in the handpiece 350, a detector 370 located in the handpiece and configured to detect the location and/or the presence of coagulated tissue, and electrical/control connections 340 between the components of the system. Some embodiments of the device of the present invention include one electromagnetic energy source capable of providing both nonablative and ablative treatments instead of two separate nonablative and ablative electromagnetic energy sources, or can include more than two electromagnetic energy sources, wherein the more than two electromagnetic energy sources include at least one source capable of delivering a nonablative treatment and at least one source capable of delivering an ablative treatment.

In the device illustrated in FIG. 3, the device is configured to deliver both the first and second optical energy-based treatments successively and coincidently using a detector to detect the presence and/or location of coagulated tissue. FIG. 3A illustrates the treatment device 300 delivering a first nonablative optical energy treatment, where the first beam of optical energy 311 impacts a portion of skin 10 creating a treatment zone containing coagulated treatment zone 20. The first beam of optical energy 311 exits the first optical energy source 310, is transmitted into the handpiece 350, and then is delivered to the skin 10 by the optical energy delivery system 360. After the first beam of optical energy 311 has coagulated the tissue in the coagulated treatment zone 20, the presence and/or location of coagulated treatment zone 20 is determined by the detector 370, and the second optical energy-based treatment can be applied such that it is coincident with the coagulated treatment zone 20.

FIG. 3B illustrates the treatment device 300 detecting the presence and/or location of coagulated tissue using the detector 370 and delivering a second ablative optical energy treatment. When the detector 370 determines the presence and/or location of coagulated tissue 20 is present in the detector's field of view 371, it communicates this to the controller, and the controller then determines when and how to fire the second beam of optical energy 321 such that the second beam of optical energy 321 will ablate at least a portion of the coagulated treatment zone 20. When fired, the second beam of optical energy 321 impacts a portion of skin 10 coincident with the portion of skin 10 that was impacted by the first treatment, thus ablating a portion 30 of the treatment zone containing coagulated treatment zone 20 and not ablating uncoagulated tissue in the region of skin 10 being treated. The second beam of optical energy 321 exits the second optical energy source 320, is transmitted into the handpiece 350, and then is delivered to the skin 10 by the optical energy delivery system 360.

Various shapes and diameters of treatment zones can be created using the device of the present invention. For example, when RF energy is used to coagulate and/or ablate the tissue, various sizes and shapes of electrodes can be used. In another example, when optical energy is used to coagulate and/or ablate the tissue, various beam sizes and shapes can be used. Examples of beam sizes for first and/or second optical energy treatments can be in the range between about 30 μm and about 2 mm, in the range between about 50 μm and about 100 μm or 1000 μm, or in the range between about 100 μm and about 500 μm, where the beam size is measured as the beam impacts the plane of the tissue to be treated. The relative sizes of the beams used to deliver the first and second optical energy treatments can be varied between the two treatments. For example, a larger beam size can be used to deliver the first optical energy treatment to create a larger coagulation zone and a somewhat smaller beam size can then be used to deliver the second optical energy treatment in order to ablate only a portion of the coagulated tissue within a treatment zone. Similarly, the beam size used to deliver the first optical energy treatment can be smaller or approximately equal to the beam size for the second optical energy treatment.

The successive coincident nonablative and ablative treatments can also be performed without a detector that detects coagulated tissue. In some embodiments, the treatments can be performed with a velocity detector that detects the motion of the handpiece relative to the skin surface and a controller that adjusts the pulse timing of an ablative energy source to create an ablative treatment zone only after the handpiece has moved a specific distance such that the ablative treatment energy is delivered to a portion of the tissue that overlaps the coagulated treatment zone 20.

Depending on the desired size and depth of the coagulated tissue and the ablated tissue within a treatment zone, the wavelength of the electromagnetic energy used can be varied. For example, when an optical energy source is used, the wavelength of the optical energy can be selected from the group consisting of between about 1,200 nm and about 20,000 nm, between about 700 nm and about 1400 nm, between about 1100 nm and about 2500 nm, between about 1280 nm and about 1350 nm, between about 1400 nm and about 1500 nm, between about 1500 nm and about 1620 nm, between about 1780 nm and 2000 nm, and combinations thereof. Wavelengths longer than 1500 nm and wavelengths with absorption coefficients in water of between about 1 cm⁻¹ and about 30 cm⁻¹ can be used if the goal is to get deep penetration with a relatively small coagulation zone. The shorter wavelengths generally have higher scattering coefficients than the longer wavelengths. The wavelength of both the first optical energy source can be strongly absorbed by water. Further, the wavelength can be in the near infrared spectrum.

Various forms of nonablative and ablative electromagnetic energy can be used in accordance with the method and device of the present invention, such as, for example, ultrasonic energy, RF energy, and optical energy. When optical energy is used, the optical energy can be coherent in nature, such as laser radiation, or non-coherent in nature, such as flashlamp radiation. Coherent optical energy can be produced by lasers, including gas lasers, dye lasers, metal-vapor lasers, fiber lasers, diode lasers, and/or solid-state lasers. The type of laser used with this invention can be selected from the group consisting of an argon ion gas laser, a carbon dioxide (CO2) gas laser, an excimer chemical laser, a dye laser, a neodymium yttrium aluminum garnet (Nd:YAG) laser, an erbium yttrium aluminum garnet (Er:YAG) laser, a holmium yttrium aluminum garnet (Ho:YAG) laser, an alexandrite laser, an erbium doped glass laser, a neodymium doped glass laser, a thulium doped glass laser, an erbium-ytterbium co-doped glass laser, an erbium doped fiber laser, a neodymium doped fiber laser, a thulium doped fiber laser, an erbium-ytterbium co-doped fiber laser, and combinations thereof. The laser can be applied in a fractional manner to produce fractional treatment. For example, the FRAXEL re:store™ laser (Reliant Technologies, Inc. Mountain View, Calif.) produces fractional treatment using an erbium-doped fiber laser operating at a wavelength that is primarily absorbed by water in tissue, at about 1550 nm.

While the method and device of the present invention can be used for medical and/or cosmetic or purposes to remodel tissue (for example, for collagen remodeling), to resurface tissue, to treat wrinkles and photoaging of the skin, and/or to remove hair, they are also suitable to treat a variety of dermatological conditions such as hypervascular lesions including port wine stains, capillary hemangiomas, cherry angiomas, venous lakes, poikiloderma of civate, angiokeratomas, spider angiomas, facial telangiectasias, telangiectatic leg veins, pigmented lesions including lentigines, ephelides, nevus of Ito, nevus of Ota, Hori's macules, keratoses pilaris; acne scars, epidermal nevus, Bowen's disease, actinic keratoses, actinic cheilitis, oral florid papillomatosis, seborrheic keratoses, syringomas, trichoepitheliomas, trichilemmomas, xanthelasma, apocrine hidrocystoma, verruca, adenoma sebacum, angiokeratomas, angiolymphoid hyperplasia, pearly penile papules, venous lakes, rosacea, etc. While specific examples of dermatological conditions are mentioned above, it is contemplated that these methods and devices can be used to treat virtually any type of dermatological condition.

Additionally, these methods and devices can be applied to other medical specialties besides dermatology. The inventions disclosed herein are also applicable to treatment of other tissues of the body. For example, the treatment of the tissue of the soft palate can also benefit from the use of this invention in order to shrink and tighten the tissue to reduce the incidence of snoring.

Although the detailed description contains many specifics, these should not be construed as limiting the scope of the invention but merely as illustrating different examples and aspects of the invention. It should be appreciated that the scope of the invention includes other embodiments not discussed in detail above. For example, the inventions disclosed herein can be generalized to RF, flashlamp, or other electromagnetic energy based treatments as well. Various other modifications, changes and variations which will be apparent to those skilled in the art may be made in the arrangement, operation and details of the methods and devices of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

All publications, patents and patent applications cited herein, whether supra or infra, are hereby incorporated by reference in their entirety.

In the specification and in the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

What is claimed is:

1. A method of treating tissue comprising:
selecting a region of tissue in need of tightening;
first, treating the region of tissue using a first form of electromagnetic energy in a manner so as to coagulate tissue within a plurality of treatment zones in the region of tissue; and
second, treating the region of tissue using a second form of electromagnetic energy in a manner so as to ablate at least a portion of the coagulated tissue from within at least a portion of the plurality of treatment zones in the region of tissue while leaving uncoagulated tissue substantially unablated,
wherein the first and second treating tighten the region of tissue.

2. The method of claim 1, wherein the first and second forms of electromagnetic energy are the same form of electromagnetic radiation.

3. The method of claim 1, wherein the first and second forms of electromagnetic energy are different forms of electromagnetic radiation.

4. The method of claim 1, wherein the first form of electromagnetic energy is nonablative and the second form of electromagnetic energy is ablative.

5. The method of claim 1, wherein the first and second forms of electromagnetic energy are both forms of optical energy.

6. The method of claim 1, wherein the first and second forms of electromagnetic energy are both forms of laser radiation.

7. The method of claim 1, wherein the first and second forms of electromagnetic energy are different wavelengths of laser radiation.

8. The method of claim 1, wherein the second treating is performed immediately following the first treating.

9. The method of claim 1, wherein the second treating is performed prior to healing of the first treating.

10. The method of claim 1, wherein the first treating comprises more than one electromagnetic energy treating.

11. The method of claim 1, wherein the second treating comprises more than one electromagnetic energy treating.

12. The method of claim 1, wherein the method further comprises the step of detecting coagulated tissue in the plurality of treatment zones.

13. The method of claim 1, wherein the method further comprises the step of determining the location of coagulated tissue in the region of tissue.

14. The method of claim 1, wherein the tissue is skin.

15. The method of claim 14, wherein the first treating coagulates at least a portion of the epidermis, and the second treating ablates at least a portion of coagulated epidermis within at least a portion of the treatment zones in the region of tissue.

16. The method of claim 14, wherein the first treating coagulates at least a portion of the dermis, and the second treating ablates at least a portion of coagulated dermis within at least a portion of the treatment zones in the region of tissue.

17. The method of claim 1, wherein the first optical energy treatment is produced by a laser selected from the group consisting of an argon ion gas laser, a carbon dioxide (CO₂) gas laser, an excimer chemical laser, a dye laser, a neodymium yttrium aluminum garnet (Nd:YAG) laser, an erbium yttrium aluminum garnet (Er:YAG) laser, a holmium yttrium aluminum garnet (Ho:YAG) laser, an alexandrite laser, an erbium doped glass laser, a neodymium doped glass laser, a thulium doped glass laser, an erbium-ytterbium co-doped glass laser, an erbium doped fiber laser, a neodymium doped fiber laser, a thulium doped fiber laser, an erbium-ytterbium co-doped fiber laser, and combinations thereof.

18. The method of claim 1, wherein the second optical energy treatment is produced by a laser selected from the group consisting of an argon ion gas laser, a carbon dioxide (CO₂) gas laser, an excimer chemical laser, a dye laser, a neodymium yttrium aluminum garnet (Nd:YAG) laser, an erbium yttrium aluminum garnet (Er:YAG) laser, a holmium yttrium aluminum garnet (Ho:YAG) laser, an alexandrite laser, an erbium doped glass laser, a neodymium doped glass laser, a thulium doped glass laser, an erbium-ytterbium co-doped glass laser, an erbium doped fiber laser, a neodymium doped fiber laser, a thulium doped fiber laser, an erbium-ytterbium co-doped fiber laser, and combinations thereof.

19. A device for tightening tissue, comprising:
a first electromagnetic energy source for providing a first electromagnetic energy treatment configured to apply the first electromagnetic energy treatment to a region of tissue in a manner so as to thermally coagulate tissue in a plurality of treatment zones in the region of tissue;
a second electromagnetic energy source for providing a second electromagnetic energy treatment configured to apply the second electromagnetic energy treatment to at least a portion of the plurality of treatment zones in the region of tissue and to thereby ablate at least a portion of the thermally coagulated tissue within;
a controller configured to control the first and second electromagnetic energy sources; and
a detector configured to detect the location and/or presence of coagulated tissue in the region of tissue and to provide feedback to the controller,
wherein the controller is configured to use the feedback from the detector to determine when and how to apply the second electromagnetic energy to ablate at least a portion of coagulated tissue in a treatment zone.

20. The device of claim 19, wherein the first and second electromagnetic energy sources are optical energy sources.

21. The device of claim 19, wherein the first and second electromagnetic energy sources are laser sources.

22. The device of claim 20, wherein a beam size of the first optical source is larger than a beam size of the second optical energy source when the beams impact the region tissue.

23. The device of claim 20, wherein a beam size of the first optical source is smaller than a beam size of the second optical energy source when the beams impact the region tissue.

24. The device of claim 20, wherein a beam size of the first and second optical energy sources are approximately equal when the beams impact the region of tissue.

25. The device of claim 20, wherein a beam size of the first optical energy source is between about 30 μm and about 2 mm.

26. The device of claim 20, wherein a beam size of the first optical energy source is between about 50 μm and about 1000 μm.

27. The device of claim 20, wherein a beam size of the first optical energy source is between about 100 μm and about 500 μm.

28. The device of claim 20, wherein a beam size of the second optical energy source is between about 30 μm and about 2 mm.

29. The device of claim 20, wherein a beam size of the second optical energy source is between about 50 μm and about 1000 μm.

30. The device of claim 20, wherein a beam size of the second optical energy source is between about 100 μm and about 500 μm.

31. The device of claim 20, wherein the wavelength of both the first optical energy source and the second optical energy source is between about 1,200 nm and about 20,000 nm.

32. The device of claim 20, wherein the wavelength of both the first optical energy source is strongly absorbed by water.

33. The device of claim 20, wherein the wavelength of the first optical energy source is in the near infrared spectrum.

34. The device of claim 20, wherein the wavelength of the first optical energy source is between about 700 nm and about 1400 nm.

35. The device of claim 20, wherein the first optical energy source is an erbium fiber laser and the second optical energy source is a carbon dioxide laser.
